(19) United States
(12) Patent Application Publication
(10) Pub. No.: US 2021/0161843 A1
(43) Pub. Date: Jun. 3, 2021

(54) PHARMACEUTICAL COMPOSITIONS OF FLURBIPROFEN AND 5-HT1 - RECEPTOR AGONISTS

(71) Applicant: SANOVEL ILAC SANAYI VE TICARET ANONIM SIRKETI, Istanbul (TR)

(72) Inventors: Ali TURKYILMAZ, Istanbul (TR); Melda MISIRLI, Istanbul (TR); Emine TUNCAY, Istanbul (TR); Ediz YILDIRIM, Istanbul (TR)

(73) Assignee: SANOVEL ILAC SANAYI VE TICARET ANONIM SIRKETI, Istanbul (TR)

(21) Appl. No.: 16/772,811

(22) PCT Filed: Dec. 13, 2018
(86) PCT No.: PCT/TR2018/050805

§ 371 Date: Jun. 15, 2020

(30) Foreign Application Priority Data
Dec. 15, 2017 (TR) 2017/20429

(51) Int. Cl. A61K 31/192; A61K 45/06; A61K 9/00

(52) U.S. Cl. CPC A61K 31/192; A61K 9/0053; A61K 45/06

(57) ABSTRACT

Pharmaceutical compositions comprising flurbiprofen or a pharmaceutically acceptable salt thereof in combination with at least one 5-HT1-receptor agonist or a pharmaceutically acceptable salt thereof. In this invention, the composition comprising flurbiprofen and 5-HT1-receptor agonists are used for treating or preventing migraine headache. Furthermore, in this invention, in order to overcome incompatibility and stability problems of these active agents, some solutions were offered, and stable pharmaceutical compositions have been developed.

### CROSS REFERENCE TO THE RELATED APPLICATIONS

This application is the national phase entry of International Application No. PCT/TR2018/050805, filed on Dec. 13, 2018, which is based upon and claims priority to Turkish Patent Application No. 2017/20429 (TR), filed on Dec. 15, 2017, the entire contents of which are incorporated herein by reference.

### TECHNICAL FIELD

The present invention relates to pharmaceutical compositions comprising flurbiprofen or a pharmaceutically acceptable salt thereof in combination with at least one 5-HT1-receptor agonist or a pharmaceutically acceptable salt thereof.

### BACKGROUND

Flurbiprofen is a propionic acid derivative, also known as NSAID (non-steroidal anti-inflammatory drug), having analgesic and anti-inflammatory activities. Its chemical structure is illustrated with Formula 1 given below.

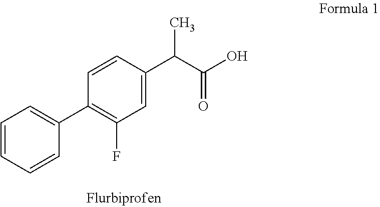 [SMILES: CC(C(=O)O)c1ccc(-c2ccccc2)c(F)c1]

Flurbiprofen is used for alleviating pain in muscle-skeleton system and joint disorders such as ankylosing spondylitis, osteoarthritis, and rheumatoid arthritis, in soft tissue injuries such as sprains and strains, in postoperative cases, and in painful and severe menstruation. Flurbiprofen is further used as a lozenge in the symptomatic amelioration of sore throats.

5-HT is a key mediator in the pathogenesis of migraine. 5-HT1-receptor agonists, commonly known as the ‘triptans’, are the mainstay for acute treatment of migraine headaches.

Generally, effective triptans may be selected from a group comprising eletriptan, sumatriptan, rizatriptan, naratriptan, zolmitriptan, frovatriptan, almotriptan, and functional analogs thereof, wherein the functional analogs have essentially the same biological activity.

Eletriptan (trade name Relpax®, used in the form of eletriptan hydrobromide) is a second generation triptan drug intended for treatment of migraine headaches. Its chemical name is 3-[[(2R)-1-methylpyrrolidin-2-yl]methyl]-5-(2-phenylsulfonylethyl)-1H-indole and its chemical structure is shown in the Formula 2.

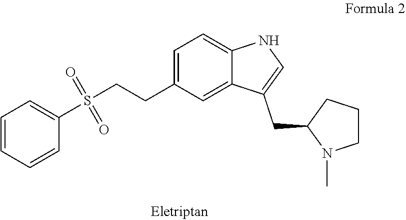 [SMILES: CN1CCC[C@@H]1Cc1c[nH]c2ccc(CCS(=O)(=O)c3ccccc3)cc12]

Eletriptan molecule and the use for the treatment of migraine is first disclosed in the patent application WO9206973.

Migraines are likely due to local cranial vasodilatation and/or to the release of sensory neuropeptides (vasoactive intestinal peptide, substance P and calcitonin gene-related peptide) through nerve endings in the trigeminal system. The therapeutic activity of eletriptan for the treatment of migraine headache is thought to be due to the agonist effects at the 5-HT1B/1D receptors on intracranial blood vessels (including the arterio-venous anastomoses) and sensory nerves of the trigeminal system which result in cranial vessel constriction and inhibition of pro-inflammatory neuropeptide release.

In prior art, patent application EP2306998 discloses a combination comprising a 5-HT1-agonist and a NSAID, but either in this application or in other patent application a pharmaceutical composition comprising flurbiprofen in combination with a 5-HT1-receptor agonist in oral administration as tablet or capsule dosage form is not specifically disclosed. Moreover, there is no combination of flurbiprofen and a 5-HT1-agonist in the market for the treatment of migraine headache.

Thus, there is a need in the art for a pharmaceutical composition or a dosage form comprising a combination of flurbiprofen and a 5-HT1-receptor agonist to achieve improved migraine headache treatment. However, some problems may occur while combining two active agents such as physicochemical incompatibility and stability problems.

### SUMMARY

According to the composition of the present invention, it is desired to provide a dosage form comprising in combination a therapeutically effective amount of flurbiprofen and 5-HT1-receptor agonists which overcomes above described problems. The main challenges when combining those molecules in the same pharmaceutical form are:

(a) to guarantee the physico-chemical compatibility between those different active ingredients and/or between the active ingredients and the excipients used; and

(b) to insure the pharmaceutical compatibility between those active ingredients regarding their stability characteristics. When flurbiprofen is combined with 5-HT1-receptor agonists, these drug substances must be released, dissolved and absorbed harmoniously. For the efficiency of the composition, an easy dissolution of both drug substances at a desired time is an important factor.

In this invention, the composition comprising flurbiprofen and 5-HT1-receptor agonists are used for treating or preventing migraine headache. It is effective at the beginning of the migraine attack.

Furthermore, in this invention, in order to overcome incompatibility and stability problems of these active agents, some solutions were offered and stable pharmaceutical compositions have been developed.

The main object of this present invention is to provide pharmaceutical compositions for the treatment of migraine headache, which has a reduced side effects with enhanced therapeutic effect.

An object of the present invention is to provide pharmaceutical compositions comprising flurbiprofen or a pharmaceutically acceptable salt thereof in combination with at least one 5-HT1-receptor agonist or a pharmaceutically acceptable salt thereof.

A further object of the present invention is to provide stable pharmaceutical compositions of flurbiprofen with at least one 5-HT1-receptor agonist.

### DETAILED DESCRIPTION OF THE EMBODIMENTS

In this invention, the pharmaceutical compositions comprise flurbiprofen or a pharmaceutically acceptable salt thereof in combination with at least one 5-HT1-receptor agonist or a pharmaceutically acceptable salt thereof and at least one pharmaceutically acceptable excipient.

Drugs of different action mechanisms can be combined. It is possible, however, to state that a combination of drugs having different action mechanisms, but showing actions on similar targets, will have absolutely positive effects.

The term “combination” means that when drugs are administered together, a combined action is obtained which is higher than the individual actions of the respective drugs when they are used separately. On the other hand, using a lower dose of each drug to be combined according to the present invention will reduce the total dosage. Put differently, the dosages have not to be relatively less in all cases, but the drugs can be dosed less frequently or this may be beneficial in reducing the recurrence rate of side effects. These are advantageous in terms of patients to be treated.

While combining more than one molecule in one dosage form is increasing the patients' quality of life, improving patient adherence, many challenges also occur such as the physicochemical compatibility between the different active agents and/or between the active agents and the excipients used; and the therapeutical compatibility between the two active agents regarding their pharmacokinetic and/or pharmaceutical properties in order that the posology of the combined formulation allows to obtain safe and efficient plasma levels of both pharmacological agents. Using different drugs may induce undesired dissolution profiles that cause undesired side effects. In this present invention, pharmaceutical dosage forms comprising flurbiprofen in combination with at least one 5-HT1-receptor agonists has been developed with safe and effective dissolution profiles for each drug molecule.

The invention comprises two active components which are administered by a single pharmaceutical composition in a pharmaceutically acceptable carrier.

According to this embodiment, the pharmaceutical composition is for oral, parenteral, intranasal, sublingual, transdermal, transmucosal, ophthalmic, intravenous, pulmonary, intramuscular or rectal administration.

In one embodiment, the pharmaceutical composition is for oral administration.

When this composition is administered to the subject orally, analgesic effect starts in a short time. 5-HT1-receptor agonists in the composition maintain the analgesic effect after the first analgesic effect is provided by flurbiprofen. Since the observable effect is provided in a short time, the patient adherence increases.

According to one embodiment of the present invention, flurbiprofen or a pharmaceutically acceptable salt thereof is present in an amount of between 10% and 80% by weight of the total composition, preferably between 10% and 50%, more preferably between 10% and 40%, the 5-HT1-receptor agonist or a pharmaceutically acceptable salt thereof is present in an amount of between 10% and 80% by weight of the total composition, preferably between 10% and 60%, more preferably between 10% and 30%.

In one embodiment, the 5-HT1-receptor agonist is selected from a group comprising eletriptan, sumatriptan, rizatriptan, naratriptan, zolmitriptan, frovatriptan or almotriptan or combinations thereof.

In one preferred embodiment, the 5-HT1-receptor agonist is selected from the group comprising eletriptan, sumatriptan, rizatriptan, zolmitriptan, frovatriptan or combinations thereof.

In one preferred embodiment, the 5-HT1-receptor agonist is eletriptan.

Suitable salts of eletriptan are selected from a group comprising acetate, aspartate, benzoate, besylate, bicarbonate/carbonate, bisulphate/sulphate, hemi sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleat, malonate, mesylate, methylsulphate, naphthylate, 2-napsylate, nicotinate, nitrate, orotate, oxalate, palmitat, pamoate, phosphate/hydrogen phosphate/dihydrogen phosphate, saccharate, stearate, succinate, tartrate, tosylat and trifluoroacetate, aluminium, arginine, benzathine, calcium, choline, diethylamine, diolamine, glycine, lysine, magnesium, meglumine, lamine, potassium, sodium, tromethamine or zinc salts.

In one preferred embodiment, the pharmaceutically acceptable salt of eletriptan is eletriptan hydrobromide.

In one preferred embodiment, the 5-HT1-receptor agonist is sumatriptan.

In one preferred embodiment, the 5-HT1-receptor agonist is rizatriptan.

In one preferred embodiment, the 5-HT1-receptor agonist is zolmitriptan.

In one preferred embodiment, the 5-HT1-receptor agonist is frovatriotan.

According to another embodiment of the present invention, the weight ratio of flurbiprofen or a pharmaceutically acceptable salt thereof to 5-HT1-receptor agonist or a pharmaceutically acceptable salt thereof is between 0.1 and 10, preferably between 0.5 and 5.0, more preferably between 1.0 and 5.0.

In one embodiment, flurbiprofen or a pharmaceutically acceptable salt thereof is present in an amount of between 0.01 mg and 300 mg, preferably between 0.1 mg and 200 mg, more preferably between 50 mg and 110 mg.

In one embodiment, 5-HT1-receptor agonist or a pharmaceutically acceptable salt thereof is present in an amount of between 0.01 mg and 100 mg, preferably between 0.1 mg and 80 mg, more preferably between 10 and 50 mg.

According to one embodiment of the invention, the pharmaceutical composition is formulated as tablets, comprising compressed tablets, coated or uncoated tablets, tablet-in-tablets, multilayer tablets, mini tablets, buccal tablets, sublingual tablets, effervescent tablets, immediate release tablets, modified release tablets, film-coated tablets, orally disintegrating tablets, gastric disintegrating tablets, pellets, effervescent compositions; capsule-in-capsules, pills, capsules, hard or soft gelatin capsules, powders, coated bead systems, granules, microspheres, ion exchange resin systems, sterile solutions or suspensions, aerosols, sprays, drops, ampoules, suppositories, parenteral systems, creams, gels, ointments, dragees, sachets, films, orally administrable films, solutions, solids, elixirs, tinctures, suspensions, syrups, colloidal dispersions or emulsions.

In one embodiment, preferably the pharmaceutical composition is in the form of a tablet or a coated tablet or a bilayer tablet or a trilayer tablet or a multilayer tablet or a capsule or encapsulated mini tablets or a capsule-in-capsule or tablet-in-tablet.

During the development study of the present invention, it has been found that flurbiprofen and 5-HT1-receptor agonists show stability and dissolution problems due to their pH inconsistency when they used together in a tablet formulation (one layer or bilayer) or in a capsule form.

In one embodiment, two active agents can be in the different forms in one dosage form.

According to this preferred embodiment, flurbiprofen or a pharmaceutically acceptable salt thereof and the 5-HT1-receptor agonist or a pharmaceutically acceptable salt thereof are in the different forms in one dosage form.

According to these embodiments, the different forms are powders, mini tablets, granules, pellets, capsules or beads.

In one embodiment, the first component which is flurbiprofen component can be in the form of powders, mini tablets, granules, pellets, capsules or beads.

In one embodiment, the second component which is 5-HT1-receptor agonist component can be in the form of powders, mini tablets, granules, pellets, capsules or beads.

In one preferred embodiment, the pharmaceutical composition is in the form of a multilayer tablet.

In one preferred embodiment, the pharmaceutical composition is in the form of a capsule comprising flurbiprofen mini tablets and 5-HT1-receptor agonists powders.

In one preferred embodiment, the pharmaceutical composition is in the form of a capsule comprising 5-HT1-receptor agonists mini tablets and flurbiprofen powders.

In one embodiment, the pharmaceutical composition is in the form of a capsule comprising 5-HT1-receptor agonists mini tablets and flurbiprofen mini tablets.

According to another embodiment of this invention, flurbiprofen mini tablets or 5-HT1-receptor agonists mini tablets or both of them comprise at least one coating layer.

The term “mini tablet”, as used herein, refers to small tablets with a diameter equal to or less than 4 mm that are typically filled into a capsule or further compressed into larger tablets. Thickness of this mini tablets equal to or less than 3 mm. The mini tablets have round shape and smooth surface to ease coating process.

Mini tablets have many advantages. Some benefits of mini-tablets include excellent size uniformity, regular shape and a smooth surface. In mini tablets, smooth surface offers an excellent substrate for coating with polymeric systems. It can be concluded that pharmaceutical mini-tablets offer several advantages when compared to single unit dosage forms and are also good substitutes for granules and pellets. They have well defined size, shape, surface, low degree of porosity and high mechanical strength. Also, while combining different mini-tablets together, incompatible drugs can be administered and excipients can be selected separately for each drug.

Suitable coating layer may also preferably be used for the protection from the moisture. It can be selected from the group comprising polyvinylpyrrolidone-vinyl acetate copolymers (Kollidon VA 64), polyvinyl alcohol-polyethylene glycol copolymers (Kollicoat IR), polyvinyl alcohol or copolymers or mixtures thereof (Opadry AMB), polymethylmetacrylate derivatives, Ethylcellulose Dispersions (Surelease), polyethylene glycol, polyvinylprolidone, polyvinylprolidone-vinyl acetate copolymer (PVP-VA) and all kinds of Opadry™, as well as pigments, dyes, titanium dioxide, iron oxide, talc or polymethylmetacrylate copolymers (Eudragit).

According to the challenges mentioned above the selection of the excipients thus very important. According to this embodiment, one or more pharmaceutically acceptable excipient is selected from buffering agents, stabilizers, antioxidants, binders, fillers, dispersing agents, lubricants, glidants, disintegrants, plasticizers, preservatives, sweeteners, flavoring agents, coloring agents, inert agent, coating agents or mixtures thereof.

Suitable buffering agents may comprise but not limited to alkali metal citrate, citric acid/sodium citrate, tartaric acid, fumaric acid, sorbic acid, citric acid, succinic acid, adipic acid, ascorbic acid, glutaric acid, potassium hydrogen tartrate, sodium hydrogen tartrate, potassium hydrogen phthalate, sodium hydrogen phthalate, potassium dihydrogen phosphate, sodium dihydrogen phosphate, disodium hydrogen phosphate, hydrochloric acid/sodium hydroxide or mixtures thereof, and preferably citric acid, fumaric acid, ascorbic acid, sodium dihydrogen phosphate, glycin, glutamic acid or mixtures thereof.

Suitable stabilizers may comprise but not limited to citric acid, fumaric acid, tartaric acid, sodium citrate, sodium benzoate, sodium dihydrogen phosphate, calcium carbonate, magnesium carbonate, arginine, lysine, meglamine, ascorbic acid, gallic acid esters or the mixtures thereof, and preferably, citric acid, fumaric acid, arginine or mixtures thereof.

Suitable antioxidants may comprise but not limited to alpha tocopherol, ascorbic acid, ascorbyl palmitate, butylhydroxyanisole (BHA), butylhydroxytoluene (BHT), erythorbic acid, monothioglycerol, potassium metabisulfite, propyl gallate, sodium ascorbate, sodium metabisulfite, sodium sulfite, thymol or mixtures thereof.

Suitable binders are selected from a group comprising microcrystalline cellulose, hydroxypropyl cellulose, sodium carboxymethyl cellulose, polyvinylpyrrolidone, carnauba wax, pullulan, glyceryl behenate, polycarbophil, polyvinyl acetate and its copolymers, cellulose acetate phthalate, hydroxypropyl starch, sugars, tragacanth gum, cetostearyl alcohol, acacia mucilage, polyethylene glycol, polyvinyl alcohol, starch, pregelatinized starch, glucose, glucose syrup, natural gums, sucrose, sodium alginate, hydroxyethyl cellulose, carboxymethyl cellulose calcium, ethyl cellulose, hydroxypropyl methyl cellulose, carboxy methyl cellulose, methyl cellulose, carrageenan, guar gum, polymethacrylates, methacrylate polymers, collagens, gelatin, agar, alginate, xanthan gum, hyaluronic acid, pectin, polysaccharides, carbomer, poloxamer, polyacrylamide, aluminium hydroxide, laponite, bentonite, polyoxyethylene-alkyl ether, polydextrose, polyethylene oxide or mixtures thereof.

In one preferred embodiment, the binders are selected from a group comprising microcrystalline cellulose, hydroxypropyl cellulose or mixtures thereof.

Suitable fillers are selected from a group comprising lactose, microcrystalline cellulose, mannitol, spray-dried mannitol, lactose monohydrate, starch, dextrose, sucrose, fructose, maltose, sorbitol, xylitol, inositol, kaolin, inorganic salts, calcium salts, polysaccharides, dicalcium phosphate, sodium chloride, dextrates, lactitol, maltodextrin, sucrose-maltodextrin mixture, trehalose, sodium carbonate, sodium bicarbonate, calcium carbonate, polyols, dextrose, maltitol or mixtures thereof.

In one preferred embodiment, the filler is lactose.

Suitable dispersing agents may comprise but not limited to calcium silicate, magnesium aluminium silicate or mixtures thereof.

Suitable lubricants are selected from a group comprising magnesium stearate, glyceryl behenate, colloidal silicon dioxide, calcium stearate, zinc stearate, talc, waxes, boric acid, hydrogenated vegetable oil, sodium chlorate, magnesium lauryl sulfate, sodium oleate, sodium acetate, sodium benzoate, polyethylene glycol, stearic acid, fatty acid, fumaric acid, glyseryl palmito sulphate, sodium stearyl fumarate, sodium lauryl sulphate or mixtures thereof.

In one preferred embodiment, the lubricant is selected from a group comprising magnesium stearate and sodium stearyl fumarate.

Suitable glidants are selected from a group comprising colloidal silicon dioxide, talc, aluminium silicate or mixtures thereof.

In one preferred embodiment, the glidant is colloidal silicon dioxide.

Suitable disintegrants are selected from a group comprising croscarmellose sodium, starch, crospovidone (cross-linked polyvinil pyrrolidone), povidone, poloxomer, low-substituted hydroxypropyl cellulose, pregelatinized starch, sodium carboxymethyl cellulose, calcium carboxymethyl cellulose, carboxymethyl cellulose, docusate sodium, guar gum, low substituted hydroxypropyl cellulose, polyacryline potassium, sodium alginate, corn starch, sodium starch glycolate, alginic acid, alginates, ion-exchange resins, magnesium aluminium silica, sodium dodesyl sulphate, poloxamer, sodium glycine carbonate, sodium lauryl sulphate or mixtures thereof.

In one preferred embodiment, the disintegrant is croscarmellose sodium.

Suitable plasticizers may comprise but not limited to polyethylene glycols of different molecular weights, propylene glycol or mixtures thereof.

Suitable preservatives may comprise but not limited to methyl paraben, propyl paraben and their salts (such as sodium, potassium), sodium benzoate, citric acid, benzoic acid, butylated hydroxytoluene or butylated hydroxyanisole, m-cresol, phenol or mixtures thereof.

Suitable sweeteners may comprise but not limited to aspartame, potassium acesulfame, sodium saccharinate, neohesperidine dihydrochalcone, sucralose, saccharin, sugars such as sucrose, glucose, lactose, fructose or sugar alcohols such as mannitol, sorbitol, xylitol, erythritol or mixtures thereof.

Suitable flavoring agents may comprise but not limited to menthol, peppermint, cinnamon, chocolate, vanillin or fruit essences such as cherry, orange, strawberry, grape, black currant, raspberry, banana, red fruits, wild berries or mixtures thereof.

Suitable coloring agents may comprise but not limited to ferric oxide, titanium dioxide, Food, Drug & Cosmetic (FD&C) dyes (such as; FD&C blue, FD&C green, FD&C red, FD&C yellow, FD&C lakes), poncau, indigo Drug & Cosmetic (D&C) blue, indigotine FD&C blue, carmoisine indigotine (indigo Carmine); iron oxides (such as; iron oxide red, yellow, black), quinoline yellow, flaming red, carmine, carmoisine, sunset yellow or mixtures thereof.

Suitable inert agents are located between the two molecules wherein the inert agent is selected from starch, lactose, D-mannitol, erythritol; lowsubstituted hydroxypropyl cellulose, hydroxypropyl cellulose, hydroxypropyl methylcellulose, polyvinylpyrrolidone, polyvinyl alcohol, methylcellulose, hydroxyethyl methylcellulose or mixtures thereof.

In one embodiment, the 5-HT1-receptor agonist is eletriptan.

In this invention, the pharmaceutical composition is for use in the treatment of migraine headache and it is effective at the beginning of the migraine attack.

### Example 1: Tablet

| Ingredients | % by weight |
|---|---|
| Flurbiprofen | 10.00-80.00 |
| Eletriptan hydrobromide | 10.00-80.00 |
| Lactose (SuperTab 11SD) | 10.00-70.00 |
| Microcrystalline cellulose PH 102 | 10.00-90.00 |
| Croscarmellose sodium | 0.10-20.00 |
| Hydroxypropyl cellulose (L-Fine | 0.10-10.00 |
| Powder) | |
| Colloidal silicon dioxide | 0.01-10.00 |
| Magnesium stearate | 0.01-10.00 |
| Total tablet | 100.00 |
| Total coated tablet | 100.00-105.00 |

The pharmaceutical compositions mentioned above are prepared by following these steps:
a. Sieving flurbiprofen, eletriptan hydrobromide, lactose (SuperTab 11SD) and hydroxypropyl cellulose (L-fine powder) and mixing for 15 minutes
b. Sieving microcrystalline cellulose PH 102 and croscarmellose sodium and adding them to the mixture and mixing for 15 minutes
c. Sieving and adding colloidal silicon dioxide to the mixture, mixing for 5 minutes
d. Sieving and adding magnesium stearate to the mixture, mixing for 5 minutes
e. Compressing the mixture to form tablets
f. Optionally, coating these tablets with Opadry AMB.

### Example 2: Tablet

| Ingredients | % by weight |
|---|---|
| Flurbiprofen | 10.00-50.00 |
| Eletriptan hydrobromide | 5.00-40.00 |
| Lactose (SuperTab 11SD) | 10.00-30.00 |
| Microcrystalline cellulose PH 102 | 10.00-60.00 |
| Croscarmellose sodium | 1.00-20.00 |
| Hydroxypropyl cellulose (L-Fine | 1.00-10.00 |
| Powder) | |
| Colloidal silicon dioxide | 0.01-1.00 |
| Magnesium stearate | 0.01-5.00 |
| Total tablet | 100.00 |
| Total coated tablet | 100.00-105.00 |

The pharmaceutical compositions mentioned above are prepared by following these steps:
a. Sieving flurbiprofen, eletriptan hydrobromide, lactose (SuperTab 11SD) and hydroxypropyl cellulose (L-fine powder) and mixing for 15 minutes
b. Sieving microcrystalline cellulose PH 102 and croscarmellose sodium and adding them to the mixture and mixing for 15 minutes
c. Sieving and adding colloidal silicon dioxide to the mixture, mixing for 5 minutes
d. Sieving and adding magnesium stearate to the mixture, mixing for 5 minutes
e. Compressing the mixture to form tablets
f. Optionally, coating these tablets with Opadry AMB.

### Example 3: Tablet

| Ingredients | % by weight |
|---|---|
| Flurbiprofen | 10.00-80.00 |
| Sumatriptan or a pharmaceutically | 10.00-80.00 |
| acceptable salt thereof | |
| Lactose (SuperTab 11SD) | 10.00-70.00 |
| Microcrystalline cellulose PH 102 | 10.00-90.00 |
| Croscarmellose sodium | 0.10-20.00 |
| Hydroxypropyl cellulose (L-Fine | 0.10-10.00 |
| Powder) | |
| Colloidal silicon dioxide | 0.01-10.00 |
| Magnesium stearate | 0.01-10.00 |
| Total tablet | 100.00 |
| Total coated tablet | 100.00-105.00 |

The pharmaceutical compositions mentioned above are prepared by following these steps:
a. Sieving flurbiprofen, sumatriptan or a pharmaceutically acceptable salt thereof, lactose (SuperTab 11SD) and hydroxypropyl cellulose (L-fine powder) and mixing for 15 minutes
b. Sieving microcrystalline cellulose PH 102 and croscarmellose sodium and adding them to the mixture and mixing for 15 minutes
c. Sieving and adding colloidal silicon dioxide to the mixture, mixing for 5 minutes
d. Sieving and adding magnesium stearate to the mixture, mixing for 5 minutes
e. Compressing the mixture to form tablets
f. Optionally, coating these tablets with Opadry AMB.

### Example 4: Tablet

| Ingredients | % by weight |
|---|---|
| Flurbiprofen | 10.00-80.00 |
| Rizatriptan or a pharmaceutically | 10.00-80.00 |
| acceptable salt thereof | |
| Lactose (SuperTab 11SD) | 10.00-70.00 |
| Microcrystalline cellulose PH 102 | 10.00-90.00 |
| Croscarmellose sodium | 0.10-20.00 |
| Hydroxypropyl cellulose (L-Fine | 0.10-10.00 |
| Powder) | |
| Colloidal silicon dioxide | 0.01-10.00 |
| Magnesium stearate | 0.01-10.00 |
| Total tablet | 100.00 |
| Total coated tablet | 100.00-105.00 |

The pharmaceutical compositions mentioned above are prepared by following these steps:
a. Sieving flurbiprofen, rizatriptan or a pharmaceutically acceptable salt thereof, lactose (SuperTab 11SD) and hydroxypropyl cellulose (L-fine powder) and mixing for 15 minutes
b. Sieving microcrystalline cellulose PH 102 and croscarmellose sodium and adding them to the mixture and mixing for 15 minutes
c. Sieving and adding colloidal silicon dioxide to the mixture, mixing for 5 minutes
d. Sieving and adding magnesium stearate to the mixture, mixing for 5 minutes
e. Compressing the mixture to form tablets
f. Optionally, coating these tablets with Opadry AMB.

### Example 5: Tablet

| Ingredients | % by weight |
|---|---|
| Flurbiprofen | 10.00-80.00 |
| Zolmitriptan or a pharmaceutically | 10.00-80.00 |
| acceptable salt thereof | |
| Lactose (SuperTab 11SD) | 10.00-70.00 |
| Microcrystalline cellulose PH 102 | 10.00-90.00 |
| Croscarmellose sodium | 0.10-20.00 |
| Hydroxypropyl cellulose (L-Fine | 0.10-10.00 |
| Powder) | |
| Colloidal silicon dioxide | 0.01-10.00 |
| Magnesium stearate | 0.01-10.00 |
| Total tablet | 100.00 |
| Total coated tablet | 100.00-105.00 |

The pharmaceutical compositions mentioned above are prepared by following these steps:
a. Sieving flurbiprofen, zolmitriptan or a pharmaceutically acceptable salt thereof, lactose (SuperTab 11SD) and hydroxypropyl cellulose (L-fine powder) and mixing for 15 minutes
b. Sieving microcrystalline cellulose PH 102 and croscarmellose sodium and adding them to the mixture and mixing for 15 minutes
c. Sieving and adding colloidal silicon dioxide to the mixture, mixing for 5 minutes
d. Sieving and adding magnesium stearate to the mixture, mixing for 5 minutes
e. Compressing the mixture to form tablets
f. Optionally, coating these tablets with Opadry AMB.

### Example 6: Tablet

| Ingredients | % by weight |
|---|---|
| Flurbiprofen | 10.00-80.00 |
| Favotriptan or a pharmaceutically | 10.00-80.00 |
| acceptable salt thereof | |
| Lactose (SuperTab 11SD) | 10.00-70.00 |
| Microcrystalline cellulose PH 102 | 10.00-90.00 |
| Croscarmellose sodium | 0.10-20.00 |
| Hydroxypropyl cellulose (L-Fine | 0.10-10.00 |
| Powder) | |
| Colloidal silicon dioxide | 0.01-10.00 |
| Magnesium stearate | 0.01-10.00 |
| Total tablet | 100.00 |
| Total coated tablet | 100.00-105.00 |

The pharmaceutical compositions mentioned above are prepared by following these steps:
a. Sieving flurbiprofen, favotriptan or a pharmaceutically acceptable salt thereof, lactose (SuperTab 11SD) and hydroxypropyl cellulose (L-fine powder) and mixing for 15 minutes
b. Sieving microcrystalline cellulose PH 102 and croscarmellose sodium and adding them to the mixture and mixing for 15 minutes
c. Sieving and adding colloidal silicon dioxide to the mixture, mixing for 5 minutes
d. Sieving and adding magnesium stearate to the mixture, mixing for 5 minutes
e. Compressing the mixture to form tablets
f. Optionally, coating these tablets with Opadry AMB.

### Example 7: Capsule Comprising Mini Tablets

| | Mini tablet 1 | % by weight |
|---|---|---|
| Flurbiprofen | Flurbiprofen | 10.00-20.00 |
| mini tablets | Lactose (SuperTab 11SD) | 5.00-15.00 |
| | Microcrystalline cellulose PH 102 | 10.00-20.00 |
| | Croscarmellose sodium | 10.00-50.00 |
| | Hydroxypropyl cellulose (L-Fine | 1.00-10.00 |
| | Powder) | |
| | Colloidal silicon dioxide | 1.00-10.00 |
| | Magnesium stearate | 0.01-1.00 |
| | Total | 100.00 |
| | Opadry AMB (optionally) | 1-10 |
| | Mini tablet 2 | % by weight |
| Eletriptan | Eletriptan hydrobromide | 20-80% |
| mini | Microcrystalline cellulose | 5.00-75.00 |
| | Lactose 11 SD | 10.00-60.00 |
| | Croscarmellose sodium | 10.00-20.00 |
| | Colloidal silicon dioxide | 1.00-10.00 |
| | Sodium stearyl fumarate | 0.01-1.00 |
| | Total | 100.00 |

The pharmaceutical compositions mentioned above are prepared by following these steps:

Preparation of Flurbiprofen Mini Tablets
a. Sieving flurbiprofen, lactose (SuperTab 11SD), microcrystalline cellulose, croscarmellose sodium and hydroxypropyl cellulose (L-fine powder) and mixing for 15 minutes
b. Sieving and adding colloidal silicon dioxide to the mixture and mixing
c. Sieving and adding magnesium stearate to the mixture and mixing
d. Compressing the mixture to form mini tablets
e. Optionally, coating these mini tablets

Preparation of Eletriptan Mini Tablets
f. Sieving eletriptan hydrobromide, lactose (SuperTab 11SD), microcrystalline cellulose, croscarmellose sodium and mixing for 15 minutes
g. Sieving and adding colloidal silicon dioxide to the mixture and mixing for 5 minutes
h. Sieving and adding sodium stearyl fumarate to the mixture and mixing
i. Compressing the mixture to form mini tablets
j. Optionally, coating these mini tablets

Preparation of Capsule Comprising Mini Tablets
k. Filling the flurbiprofen mini tablets and the eletriptan mini tablets into capsules

### Example 8: Capsule Comprising Mini Tablets and Granules

| | Mini tablet | % by weight |
|---|---|---|
| Flurbiprofen | Flurbiprofen | 10.00-20.00 |
| mini tablets | Lactose (SuperTab 11SD) | 5.00-15.00 |
| | Microcrystalline cellulose PH 102 | 10.00-20.00 |
| | Croscarmellose sodium | 10.00-50.00 |
| | Hydroxypropyl cellulose (L-Fine | 1.00-10.00 |
| | Powder) | |
| | Colloidal silicon dioxide | 1.00-10.00 |
| | Magnesium stearate | 0.01-1.00 |
| | Total | 100.00 |
| | Opadry AMB (optionally) | 1-10 |
| | Granule | % by weight |
| Eletriptan | Eletriptan hydrobromide | 20-80% |
| granules | Microcrystalline cellulose | 5.00-75.00 |
| | (PH102) | |
| | Lactose 11 SD | 10.00-60.00 |
| | Croscarmellose sodium | 10.00-20.00 |
| | Colloidal silicon dioxide | 1.00-10.00 |
| | Sodyum stearyl fumarate | 0.01-1.00 |
| | Total | 100.00 |
| | Opadry AMB (optionally) | 1-10 |

Preparation of flurbiprofen mini tablets
a. Sieving flurbiprofen, lactose (SuperTab 11SD), microcrystalline cellulose (PH102), croscarmellose sodium and hydroxypropyl cellulose (L-fine powder) and mixing for 15 minutes
b. Sieving and adding colloidal silicon dioxide to the mixture and mixing
c. Sieving and adding magnesium stearate to the mixture and mixing
d. Compressing the mixture to form mini tablets
e. Optionally, coating these mini tablets

Preparation of Eletriptan Granules
f. Sieving eletriptan hydrobromide, lactose (SuperTab 11SD), microcrystalline cellulose, croscarmellose sodium and mixing for 15 minutes
g. Sieving and adding colloidal silicon dioxide to the mixture and mixing for 5 minutes
h. Sieving and adding sodium stearyl fumarate to the mixture and mixing for 5 minutes to form granules

Preparation of Capsule Comprising Mini Tablets
i. Filling the flurbiprofen mini tablets and eletriptan granules into capsules.

1. A pharmaceutical composition comprising flurbiprofen or a pharmaceutically acceptable salt thereof in combination with a 5-HT 1-receptor agonist or a pharmaceutically acceptable salt thereof and at least a pharmaceutically acceptable excipient.

2. The pharmaceutical composition according to claim 1, wherein the 5-HT1-receptor agonist is selected from a group comprising eletriptan, sumatriptan, rizatriptan, naratriptan, zolmitriptan, frovatriptan or almotriptan or combinations thereof.

3. The pharmaceutical composition according to claim 1, wherein the pharmaceutical composition is for oral, parenteral, intranasal, sublingual, transdermal, transmucosal, ophthalmic, intravenous, pulmonary, intramuscular or rectal administration.

4. The pharmaceutical composition according to claim 3, wherein the composition is for oral administration.

5. The pharmaceutical composition according to claim 1, wherein a weight ratio of the flurbiprofen or the pharmaceutically acceptable salt thereof to the 5-HT1-receptor agonist or the pharmaceutically acceptable salt thereof is between 0.1 and 10.

6. The pharmaceutical composition according to claim 5, wherein the flurbiprofen or the pharmaceutically acceptable salt thereof is present in an amount of between 10% and 80% by weight of the total composition.

7. The pharmaceutical composition according to claim 5, wherein the 5-HT1-receptor agonist or the pharmaceutically acceptable salt thereof is present in an amount of between 10% and 80% by weight of the total composition.

8. The pharmaceutical composition according to claim 5, wherein the flurbiprofen or the pharmaceutically acceptable salt thereof is present in an amount of between 0.01 mg and 300 mg.

9. The pharmaceutical composition according to claim 5, wherein the 5-HT1-receptor agonist or the pharmaceutically acceptable salt thereof is present in an amount of between 0.01 mg and 100 mg.

10. The pharmaceutical composition according to claim 1, wherein the pharmaceutical composition is formulated as tablets, comprising compressed tablets, coated or uncoated tablets, tablet-in-tablets, multilayer tablets, mini tablets, buccal tablets, sublingual tablets, effervescent tablets, immediate release tablets, modified release tablets, film-coated tablets, orally disintegrating tablets, gastric disintegrating tablets; capsule-in-capsules, pellets, effervescent compositions, pills, capsules, hard or soft gelatin capsules, powders, coated bead systems, granules, microspheres, ion exchange resin systems, sterile solutions, suspensions, aerosols, sprays, drops, ampoules, suppositories, parenteral systems, creams, gels, ointments, dragees, sachets, films, orally administrable films, solutions, solids, elixirs, tinctures, suspensions, syrups, colloidal dispersions or emulsions.

11. The pharmaceutical composition according to claim 10, wherein the pharmaceutical composition is in a form of a tablet or a coated tablet or a bilayer tablet or a trilayer tablet or a multilayer tablet or a capsule or encapsulated mini tablets or a capsule-in-capsule or tablet-in-tablet.

12. The pharmaceutical composition according to claim 11, wherein the flurbiprofen or the pharmaceutically acceptable salt thereof and the 5-HT1-receptor agonist or the pharmaceutically acceptable salt thereof are in a plurality of different forms in one dosage form.

13. The pharmaceutically composition according to claim 12, the plurality of different forms are powders, mini tablets, granules, pellets, capsules or beads.

14. The pharmaceutical composition according to claim 1, for use in a treatment of migraine headache.

15. The pharmaceutical composition according to claim 5, wherein a weight ratio of the flurbiprofen or the pharmaceutically acceptable salt thereof to the 5-HT1-receptor agonist or the pharmaceutically acceptable salt thereof is between 1.0 and 5.0.

16. The pharmaceutical composition according to claim 6, wherein the flurbiprofen or the pharmaceutically acceptable salt thereof is present in an amount of between 10% and 40% by weight of the total composition.

17. The pharmaceutical composition according to claim 7, wherein the 5-HT1-receptor agonist or the pharmaceutically acceptable salt thereof is present in an amount of between 10% and 30% by weight of the total composition.

18. The pharmaceutical composition according to claim 8, wherein the flurbiprofen or the pharmaceutically acceptable salt thereof is present in an amount of between 50 mg and 110 mg.

19. The pharmaceutical composition according to claim 9, wherein the 5-HT1-receptor agonist or the pharmaceutically acceptable salt thereof is present in an amount of between 10 mg and 50 mg.
